(19) United States
(12) Patent Application Publication
(10) Pub. No.: US 2025/0064728 A1
(43) Pub. Date: Feb. 27, 2025

(54) HYDROGEL-FORMING SYSTEMS INCLUDING BIOLOGICALLY INNOCUOUS BUFFER

(71) Applicant: Boston Scientific Scimed, Inc., Maple Grove, MN (US)

(72) Inventors: Nicolas Ball-Jones, Minneapolis, MN (US); Joseph Thomas Delaney, JR., Minneapolis, MN (US); Yen-Hao Hsu, Shrewsbury, MA (US); Cristian Parisi, Boston, MA (US)

(73) Assignee: Boston Scientific Scimed, Inc., Maple Grove, MN (US)

(21) Appl. No.: 18/805,710

(22) Filed: Aug. 15, 2024

Related U.S. Application Data
(60) Provisional application No. 63/578,295, filed on Aug. 23, 2023.

(51) Int. Cl. A61K 9/06; A61K 9/00; A61K 47/42

(52) U.S. Cl. CPC A61K 9/06; A61K 9/0024; A61K 47/42

(57) ABSTRACT

In some aspects, the present disclosure provides a system for forming a hydrogel composition that comprises (a) a first reservoir containing a reactive polymer that comprises a plurality of first reactive groups; (b) a second reservoir containing a multifunctional compound that comprises a plurality of second reactive groups, the second reactive groups reacting with the first reactive groups to form covalent bonds at a pH greater than 7.4; and (c) a third reservoir containing a biologically innocuous buffer having a pH in a range of 8 to 12. Other aspects of the present disclosure pertain methods of treatment using such systems.

### CROSS-REFERENCE TO RELATED APPLICATIONS

This application claims the benefit of U.S. Provisional Patent Application Ser. No. 63/578,295 filed on Aug. 23, 2023, the disclosure of which is incorporated herein by reference.

### FIELD

The present disclosure relates to systems for use in forming hydrogels and to methods of making hydrogels using such systems.

### BACKGROUND

SpaceOAR® is a rapid crosslinking hydrogel that polymerizes in vivo and is based on a multi-arm polyethylene glycol (PEG) polymer functionalized with succinimidyl glutarate as activated end groups which further react with trilysine to form crosslinks. This product has been used clinically in prostate cancer therapy. A further improvement based on this structure is that a portion the succinimidyl glutarate end groups have been functionalized with 2,3,5-triiiodobenzamide groups, providing radiopacity. This hydrogel, known by the trade name of SpaceOAR Vue®, is the radiopaque version of SpaceOAR® for prostate medical applications. During application, two solutions (a) a solution of trilysine and multi-arm-PEG in a phosphate buffer and (b) an accelerant solution comprising sodium tetraborate decahydrate are mixed together via a Y-connector and simultaneously injected into a space between the rectum and prostate via an 18-gauge needle. When mixed, the solutions crosslink to form a solid hydrogel within seconds. This hydrogel spacer persists for approximately 3 months and then gradually dissipates via hydrolysis. Sodium tetraborate decahydrate, however, has the following GHS-US classification: Reproductive toxicity (Category 1B) H360 May damage fertility or the unborn child. While not of significant concern with prostrate radiotherapy treatment, which may render the male recipient infertile, for broader use of SpaceOAR® beyond prostate treatment, a more biologically innocuous buffer would be desirable.

### SUMMARY

In some aspects, the present disclosure provides a system for forming a hydrogel composition that comprises (a) a first reservoir containing a reactive polymer that comprises a plurality of first reactive groups; (b) a second reservoir containing a multifunctional compound that comprises a plurality of second reactive groups, the second reactive groups reacting with the first reactive groups to form covalent bonds at a pH greater than 7.4; and (c) a third reservoir containing a biologically innocuous buffer having a pH in a range of 8 to 12.

In some embodiments, the first reactive groups are electrophilic groups and the second reactive groups are nucleophilic groups. In some of these embodiments, the first reactive groups are cyclic imide ester groups and the second reactive groups are amine groups.

In some embodiments, which can be used in conjunction with the above aspects and embodiments, the biologically innocuous buffer is selected from organic buffers, inorganic buffers, and non-nucleophilic polymeric buffers.

In some embodiments, which can be used in conjunction with the above aspects and embodiments, the biologically innocuous buffer is an organic buffer that comprises a secondary or tertiary amine and does not comprise a primary amine.

In some embodiments, which can be used in conjunction with the above aspects and embodiments, the biologically innocuous buffer is an inorganic buffer that selected from a bicarbonate buffer, a phosphate buffer, or a nanoclay-based buffer.

In some embodiments, which can be used in conjunction with the above aspects and embodiments, the reactive polymer is a reactive multi-arm polymer that comprises a plurality of hydrophilic polymer arms comprising hydrophilic polymer segments and first reactive groups as end groups. In some of these embodiments, the hydrophilic polymer segments are selected from polyalkylene oxide segments, polyester segments, polyoxazoline segments, polydioxanone segments, and polypeptide segments. In some of these embodiments, the first reactive groups are linked to the hydrophilic polymer segments by a hydrolysable ester.

In some embodiments, which can be used in conjunction with the above aspects and embodiments, the multifunctional compound comprises a plurality of —(CH₂)ₓ—NH₂ groups where x is 0, 1, 2 3, 4, 5 or 6.

In some embodiments, which can be used in conjunction with the above aspects and embodiments, the multifunctional compound comprises a peptide oligomer that comprises from 2 to 10 lysine and/or ornithine amino acid residues.

In some embodiments, which can be used in conjunction with the above aspects and embodiments, the first reservoir contains a dry composition containing the reactive polymer, wherein the second reservoir contains a first fluid composition containing the multifunctional compound, and wherein the third reservoir contains a second fluid composition containing the biologically innocuous buffer. In some of these embodiments, the first fluid composition containing the multifunctional compound is buffered to a pH ranging from 3 to 5.

In some embodiments, which can be used in conjunction with the above aspects and embodiments, the system further comprises a delivery device. In some of these embodiments, the delivery device comprises a double barrel syringe.

In some embodiments, which can be used in conjunction with the above aspects and embodiments, the system further comprises one or more of a needle, catheter tube or cannula.

In other aspects, the present disclosure pertains to methods of treating a subject using a system in accordance with any of the above aspects and embodiments, the method comprising: (a) mixing the first fluid composition containing the multifunctional compound in the second reservoir with the reactive polymer in the first reservoir to form a precursor fluid; (b) mixing the precursor fluid with the second fluid composition containing the biologically innocuous buffer to form a fluid mixture; and (c) injecting the fluid mixture into or onto tissue of the subject whereupon a hydrogel forms in or on the tissue.

In some embodiments, the fluid mixture is injected from a device that simultaneously mixes the precursor fluid and the second fluid composition.

In some embodiments, the precursor fluid has a pH ranging from 3 to 5.

In some embodiments, the fluid mixture has a pH ranging from 7.4 to 12.

The above and other aspects, embodiments, features and benefits of the present disclosure will be readily apparent from the following detailed description.

### BRIEF DESCRIPTION OF THE DRAWINGS

FIG. 1 schematically illustrates a delivery device, in accordance with an embodiment of the present disclosure.

FIG. 2 schematically illustrates a delivery device, in accordance with another embodiment of the present disclosure.

### DETAILED DESCRIPTION

In some aspects, the present disclosure pertains to hydrogel-forming system that comprises (a) a reactive polymer that comprises a plurality of first reactive groups, (b) a multifunctional compound that comprises a plurality of second reactive groups that are reactive with the first reactive groups, wherein reactivity between the first and second reactive groups increase with increasing pH and (c) a biologically innocuous buffer having a pH in a range of 7.4-12.

When the first reactive groups of the reactive polymer and the second reactive groups of the multifunctional compound react with one another, a crosslinked product, in particular, a hydrogel, is formed. As used herein, a hydrogel refers to a water-containing, three-dimensional crosslinked polymer network. The reactive polymer and the multifunctional compound may be water soluble.

In some embodiments, a precursor fluid containing the reactive polymer and the multifunctional compound is provided, the precursor fluid having a pH where crosslinking between the reactive polymer and the multifunctional compound is suppressed. Then, when crosslinking is desired, the biologically innocuous buffer is mixed with the precursor fluid to form a fluid crosslinking mixture that has an increased pH value where crosslinking between the reactive polymer and the multifunctional compound is accelerated, forming a hydrogel product.

In some embodiment, the pH of the fluid crosslinking mixture is at least 2 pH units greater than the pH over the precursor fluid.

In some embodiments, the precursor fluid will not form a hydrogel product at room temperature for at least 60 minutes, if at all, whereas after the addition of the biologically innocuous buffer, a hydrogel is formed in less than one minute.

In some embodiments, the pH of the biologically innocuous buffer may range from 7.4 to 12, more typically from 8.5 to 11, even more typically from 9 to 10. In some embodiments, the pH of the precursor fluid may range from 2 to 6, more typically from 3 to 5. In some embodiments, the pH of the crosslinking fluid may range from 7.4 to 12, more typically from 8 to 11, even more typically from 9 to 11.

In some embodiments, the rate of reaction between the reactive polymer and the multifunctional compound in the fluid crosslinking mixture is at least 1000 times greater than the rate of reaction between the reactive polymer and the multifunctional compound in the precursor fluid before the addition of the biologically innocuous buffer. For example and without wishing to be bound by theory, in the specific case where the first reactive groups are succinimide ester groups and the second reactive groups are primary amine groups, based on the Henderson-Hasselbalch equation, the amine groups are activated for reaction with the succinimide ester groups on a gradient scale based on the pH, where each increase in pH by 1 unit leads to 10× greater expected reaction rate.

In some embodiment, a hydrogel is formed that, when formed in a container, such as a test tube, will remain in the container when inverted. In some embodiment, a hydrogel is formed that maintains a three-dimensional shape (e.g., a spheroidal shape, a rectangular shape, etc.) when placed on a flat surface.

Examples of biologically innocuous buffers include inorganic buffers, organic buffers, and non-nucleophilic polymeric buffers. Particular examples of inorganic buffers include bicarbonate buffer (9.2-10.6=pH), phosphate buffer (5.8-8.0=pH) (e.g., phosphate buffered saline), and nanoclay-containing buffers (e.g., sacrificially buffering to pH=˜9.5). One beneficial nanoclay for use herein is Laponite®, a smectite clay, which is composed of layered synthetic silicate synthesized from inorganic mineral salts, is characterized by the empirical formula Na_{0.7}Si₈Mg_{5.5}Li_{0.3}O₂₀(OH)₄, has a disk-shaped geometry of 20-50 nm diameter and 1-2 nm thickness, and exhibits a dual charge distribution, i.e., a negative charge on surface of the particle and a positive charge along the edges. Laponite® nanoclay, is composed of layered structures of octahedral magnesium and lithium ions sandwiched between tetrahedral silicon ions. The isoelectric point of Laponite® nanoclay is pH˜10, exhibiting a stable structure at higher pH, while exhibiting chemical dissolution of individual particles at lower pH (<9). Laponite® nanoclay demonstrates notable pH-buffering capabilities. When nanoclay is dispersed in water below pH˜9, it dissociates into non-toxic ionic products (Na⁺, Mg²⁺, Si(OH)₄, Li⁺), with OH⁻ ions dissociating from the nanoclay to re-establish basic pH. For further information on Laponite®, see, e.g., Akhilesh K. Gaharwar, et al., “Two-Dimensional Nanoclay for Biomedical Applications: Regenerative Medicine, Therapeutic Delivery, and Additive Manufacturing”. Adv Mater. 2019 June; 31(23): e1900332, published online 2019 Apr. 3. doi: 10.1002/adma.201900332 and the references cited therein. The concentration of the inorganic components of the inorganic biologically innocuous buffers of the present disclosure may range, for example, from 0.1 to 5, among other possible ranges.

Organic buffers include those that have either sterically encumbered amines or secondary/tertiary amines (to inhibit or prevent reaction with the first reactive groups of the reactive polymer, including cyclic imide ester groups as described below). Particular examples of organic biologically innocuous buffers include Bis-TRIS propane (6.3-9.5=pH; CAS: 64431-96-5),

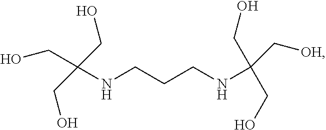 [SMILES: OCC(CO)(CO)NCCCNC(CO)(CO)CO]

BES (6.4-7.8=pH; CAS: 10191-18-1),

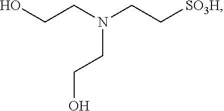 [SMILES: O=S(=O)(O)CCN(CCO)CCO]

MOPS (6.5-7.9=pH; CAS: 1132-61-2),

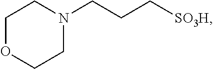 [SMILES: O=S(=O)(O)CCCN1CCOCC1]

TES (6.8-8.2=pH; CAS: 7365-44-8),

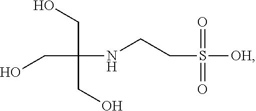 [SMILES: O=S(=O)(O)CCNC(CO)(CO)CO]

HEPES (6.8-8.2=pH; CAS: 7365-45-9),

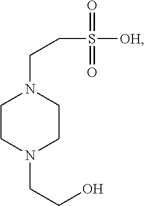 [SMILES: O=S(=O)(O)CCN1CCN(CCO)CC1]

MOBS (6.9-8.3=pH; CAS: 115724-21-5),

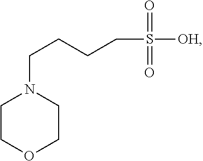 [SMILES: O=S(=O)(O)CCCCN1CCOCC1]

TAPSO (7.0-8.2=pH; CAS: 68399-81-5),

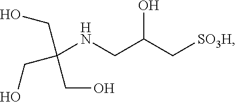 [SMILES: O=S(=O)(O)CC(O)CNC(CO)(CO)CO]

Tris/Trizma (7.0-9.0=pH; CAS: 77-86-1),

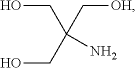

POPSO (7.2-8.5=pH; CAS: 68189-43-5),

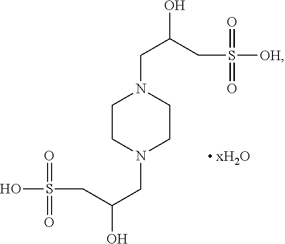 [SMILES: O=S(=O)(O)CC(O)CN1CCN(CC(O)CS(=O)(=O)O)CC1]

EPPS (7.3-8.7=pH; CAS: 16052-06-5),

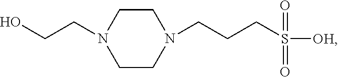 [SMILES: O=S(=O)(O)CCCN1CCN(CCO)CC1]

Tricene (7.4-8.8=pH; CAS: 5704-04-1),

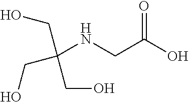 [SMILES: O=C(O)CNC(CO)(CO)CO]

Bicine (7.6-9.0=pH; CAS: 150-25-4),

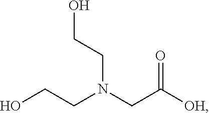 [SMILES: O=C(O)CN(CCO)CCO]

HEPBS (7.6-9.0=pH; CAS: 161308-36-7),

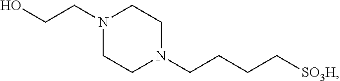 [SMILES: O=S(=O)(O)CCCCN1CCN(CCO)CC1]

TAPS (7.7-9.1=pH; CAS: 29915-38-6),

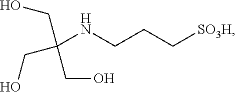 [SMILES: O=S(=O)(O)CCCNC(CO)(CO)CO]

(7.8-9.7=pH; CAS: 115-69-5),

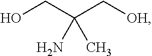

AMPSO (8.3-9.7=pH; CAS:68399-79-1),

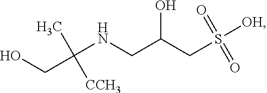

CHES (8.6-10.0=pH; CAS: 103-47-9),

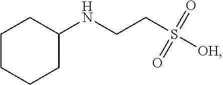 [SMILES: O=S(=O)(O)CCNC1CCCCC1]

CAPSO (8.9-10.3=pH; CAS: 73463-39-5),

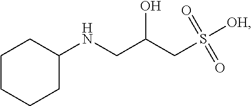 [SMILES: O=S(=O)(O)CC(O)CNC1CCCCC1]

CAPS (9.7-11.1=pH; CAS:1135-40-6),

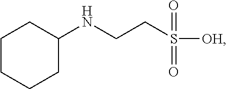 [SMILES: O=S(=O)(O)CCNC1CCCCC1]

CABS (10-11.4=pH; CAS: 161308-34-5), and Pro-Pro,

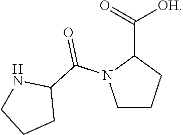 [SMILES: O=C(O)C1CCCN1C(=O)C1CCCN1]

The concentration of the organic components of the organic biologically innocuous buffers of the present disclosure may range, for example, from 0.1 to 70%, among other possible ranges.

Non-nucleophilic polymeric buffers include polyarginine (˜12=pH) and poly(dimethylaminoethylmethacrylate) (pKa=8.1-9.8 depending on degree of polymerization). The concentration of the polymers of the non-nucleophilic polymeric biologically innocuous buffers of the present disclosure may range, for example, from 0.1 to 50% among other possible ranges. The molecular weight of the polymers in the non-nucleophilic polymeric biologically innocuous buffers of the present disclosure may range, for example, from 0.5 kg/mol to 500 kg/mol among other possible ranges. The osmolarity of the final system (precursor solution combined with non-nucleophilic polymeric biologically innocuous buffer) of the present disclosure may range, for example, from 100 to 500, among other possible ranges.

As previously indicated, in addition to a biologically innocuous buffer, the hydrogel-forming systems of the present disclosure also comprise a reactive polymer that comprises a plurality of first reactive groups and a multifunctional compound that comprises a plurality of second reactive groups that are reactive with the first reactive groups.

In some embodiments, the first reactive groups are electrophilic groups and the second reactive groups are nucleophilic groups. In some embodiments, the first reactive groups are nucleophilic groups and the second reactive groups are electrophilic groups.

Reactive polymers for use in the present disclosure include reactive multi-arm polymers that comprise a plurality of polymer arms linked to a core region, at least a portion of the arms comprising a hydrophilic polymer segment. One end of the hydrophilic polymer segment is covalently linked to the core region and an opposite end of the hydrophilic polymer segment is covalently linked to a first reactive group.

In certain embodiments, at least a portion of the polymer arms comprise a hydrophilic polymer segment that has first and second ends, the first end of the hydrophilic polymer segment covalently linked to the core region, a cyclic anhydride residue having first and second ends, the first end of the cyclic anhydride residue covalently linked to the second end of the hydrophilic polymer segment, and a first reactive group that is covalently linked to the second end of the cyclic anhydride residue.

Reactive polymers in accordance with the present disclosure include reactive multi-arm polymers having from 3 to 100 arms, for example ranging anywhere from 3 to 4 to 5 to 6 to 7 to 8 to 10 to 12 to 15 to 20 to 25 to 50 to 75 to 100 arms (in other words, having a number of arms ranging between any two of the preceding values).

Preferred first reactive groups are electrophilic groups. Electrophilic groups may be selected, for example, from cyclic imide ester groups, such as succinimide ester groups,

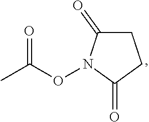

maleimide ester groups, glutarimide ester groups, diglycolimide ester groups, phthalimide ester groups, and bicyclo[2.2.1]hept-5-ene-2,3-dicarboxylic acid imide,

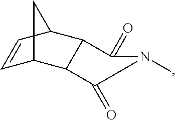

ester groups, imidazole ester groups, imidazole carboxylate groups and benzotriazole ester groups, among other possibilities.

The electrophilic groups may be linked to the hydrophilic polymer segment through any suitable linking group, which may be selected, for example, from a linking group that comprises an alkyl group, a linking group that comprises an ether group, a linking group that comprises an ester group, a linking group that comprises an amide group, a linking group that comprises an amine group, a linking group that comprises a carbonate group, or a linking group that comprises a combination of two or more of the foregoing groups, among others. In certain embodiments, the linking group comprises a hydrolysable ester group.

Hydrophilic polymer segments for the polymer arms can be selected from a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain between 10 and 1000 monomer units.

In certain embodiments, the core region comprises a residue of a polyol comprising three or more hydroxyl groups, which is used to form the polymer arms. In certain beneficial embodiments, the core region comprises a residue of a polyol that contains from 3 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

In other embodiments, the core region comprises a silsesquioxane, which is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three 0 atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

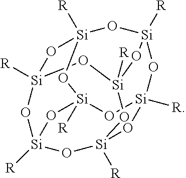

In the present disclosure, the R groups comprise the polymer arms described herein.

Reactive multi-arm polymers in accordance with the present disclosure can be formed from hydroxy-terminated precursor multi-arm polymers having arms that comprise one or more hydroxyl end groups. In some of these embodiments, the hydroxy-terminated precursor multi-arm hydrophilic polymer may be reacted with a cyclic anhydride to form an acid-end-capped precursor polymer. For example, terminal hydroxyl groups of the hydrophilic segments may be reacted with a cyclic anhydride (e.g., a glutaric anhydride compound, a succinic anhydride compound, a malonic anhydride compound, an adipic anhydride compound, a diglycolic anhydride compound, etc.) to form an acid-end-capped segment such as a glutaric-acid-end-capped segment, a succinic-acid-end-capped segment, a malonic-acid-end-capped segment, an adipic-acid-end-capped segment, a diglycolic-acid-end-capped segment, and so forth.

The preceding cyclic anhydrides, among others, may be reacted with a hydroxy-terminated precursor multi-arm hydrophilic polymer under basic conditions to form a carboxylic-acid-terminated precursor polymer comprising a carboxylic acid end group that is linked to a hydrophilic polymer segment through a hydrolysable ester group.

A reactive group may then be linked to the carboxylic-acid-terminated precursor polymer.

In some embodiments, an electrophilic group may be linked to the carboxylic-acid-terminated precursor polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl′propyl)carbodiimide (EDC), N-hydroxybenzotriazole (HOBt), BOP reagent, and/or another coupling agent) to form a reactive cyclic imide ester (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

Multifunctional compounds comprise a plurality of second reactive groups for use in the present disclosure include multifunctional compound that comprises a plurality of nucleophilic groups, such as polyamine compounds. In general, polyamine compounds suitable for use in the present disclosure include, for example, small molecule polyamines (e.g., containing at least two amine groups, for instance, from 3 to 20 amine groups or more in certain embodiments), polymers having amine side groups, and branched polymers having amine end groups, including dendritic polymers having amine end groups. Polyamine compounds suitable for use in the present disclosure include those that comprise a plurality of —(CH₂)ₓ—NH₂ groups where x is 0, 1, 2, 3, 4, 5 or 6.

Polyamine compounds which may be used as the multifunctional compound include polyamine compounds that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.). Polyamine compounds which may be used as the multifunctional compound further include ethylenetriamine, diethylene triamine, hexamethylenetriamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE® polyetheramines available from Huntsman Corporation, chitosan and derivatives thereof, poly(vinyl amine), and poly(allyl amine), among others.

In certain beneficial embodiments, the reactive polymers and/or multifunctional compounds of the present disclosure comprise one or more covalently linked radiopaque moieties, for example, bromine or iodine groups. For instance, a reactive polymer and/or a polyamine compound such as those described above, among others, may be linked to an iodine-containing moiety through a suitable linking group, for example, a linking group that comprises an amide group, a linking group that comprises an amine group, a linking group that comprises a carbonate group, or a linking group that comprises a combination of two or more of the foregoing groups, among others. Examples of such iodine-containing moieties include aromatic moieties that comprise a monocyclic or multicyclic aromatic structure, such as a benzene group or a naphthalene group, that is substituted with the following: one or more radiopaque functional groups, for example, one or more iodine groups and, optionally, a plurality of hydrophilic functional groups, for example, hydrophilic functional groups selected from one or more of hydroxyl groups, C₁-C₄-hydroxyalkyl groups, C₁-C₄-aminoalkyl groups or C₁-C₄-carboxyalkyl groups.

In various embodiments, the crosslinked hydrogels of the present disclosure are visible under fluoroscopy. In various embodiments, such crosslinked hydrogels have a radiopacity that is greater than 250 Hounsfield units (HU), beneficially anywhere ranging from 250 HU to 500 HU to 750 HU to 1000 HU or more (in other words, ranging between any two of the preceding numerical values).

As previously noted, in various aspects, the present disclosure pertains to hydrogel-forming system that comprises (a) a reactive polymer that comprises a plurality of first reactive groups, (b) a multifunctional compound that comprises a plurality of second reactive groups that are reactive with the first reactive groups, wherein reactivity between the first and second reactive groups increase with increasing pH and (c) a biologically innocuous buffer.

In some embodiments, a precursor fluid containing the reactive polymer and the multifunctional compound is provided, the precursor fluid having a pH where crosslinking between the reactive polymer and the multifunctional compound is suppressed. Then, when crosslinking is desired, the biologically innocuous buffer is added to precursor fluid, thereby increasing the pH to a value where crosslinking between the reactive polymer and the multifunctional compound is accelerated, forming a hydrogel product.

Such systems can be used to form crosslinked hydrogels, either in vivo or ex vivo. For example, crosslinked hydrogels may be formed in vivo (e.g., using a delivery device like that described below), or crosslinked hydrogels may be formed ex vivo and subsequently administered to a subject. The crosslinked hydrogels can be used in a wide variety of biomedical applications, including implants, medical devices, and pharmaceutical compositions.

In some embodiments, a system is provided that comprises (a) a first composition that comprises a reactive polymer as described herein, (b) a second composition that comprises a multifunctional compound as described herein, and (c) a third composition that comprises a biologically innocuous buffer as described herein. The system is configured to combine the reactive polymer, the multifunctional compound, and the biologically innocuous buffer under conditions such that covalent crosslinks are formed between the reactive polymer and the multifunctional compound.

The first composition may be a fluid composition comprising the reactive polymer or a dry composition that comprises the reactive polymer, to which a suitable fluid such as water for injection, saline, etc. can be added to form a fluid composition. In addition to the reactive polymer, the first composition may further comprise additional agents, including therapeutic agents, imaging agents, colorants, tonicity adjusting agents, suspension agents, wetting agents, and pH adjusting agents as described below.

The second composition may be a fluid composition comprising the multifunctional compound or a dry composition that comprises the multifunctional compound, to which a suitable fluid such as water for injection, saline, etc. can be added to form a fluid composition. In addition to the multifunctional compound, the second composition may further comprise additional agents, including therapeutic agents, imaging agents, colorants, tonicity adjusting agents, suspension agents, wetting agents, and pH adjusting agents as described below.

The third composition may be a third fluid composition that comprises the biologically innocuous buffer or a third dry composition, to which a suitable fluid such as water for injection, saline, etc. can be added to form a third fluid composition that comprises the biologically innocuous buffer. In some embodiments, the third composition may further comprise additional agents, including therapeutic agents, imaging agents, colorants, tonicity adjusting agents, suspension agents, wetting agents, and pH adjusting agents as described below.

In a particular embodiment, the first composition comprises a dry composition that comprises the reactive polymer and the second composition is a fluid composition comprising the multifunctional compound that is buffered to an acidic pH. The second composition may then be mixed with the first composition to provide a precursor fluid composition that is buffered to an acidic pH and comprises the multifunctional compound and the reactive polymer. In a particular example, a syringe may be provided that contains a fluid composition comprising the multifunctional compound that is buffered to an acidic pH, and a vial may be provided that comprises dry composition (e.g., a powder) that comprises the reactive polymer. The syringe may then be used to inject the fluid composition comprising the multifunctional compound that is buffered to an acidic pH into the vial containing the dry composition that comprises the reactive polymer to form a precursor fluid composition that is buffered to an acidic pH and contains the multifunctional compound and the reactive polymer, which can be withdrawn back into the syringe for administration.

The third composition may be a fluid accelerant composition that comprises the biologically innocuous buffer or may be a dry composition, to which a suitable fluid such as water for injection, saline, etc. is added to form a fluid accelerant composition.

A precursor fluid composition that is buffered to an acidic pH and comprises the multifunctional compound and the reactive polymer as described above, and a fluid accelerant composition that comprises a biologically innocuous buffer as described above, may be combined form crosslinked hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a precursor fluid composition that is buffered to an acidic pH and comprises the multifunctional compound and the reactive polymer as described above and a second reservoir that contains a fluid accelerant composition that comprises a biologically innocuous buffer as described above.

During operation, the precursor fluid composition and fluid accelerant composition are dispensed from the first and second reservoirs and combined, whereupon the multifunctional compound and the reactive polymer react and crosslink with one another to form a hydrogel.

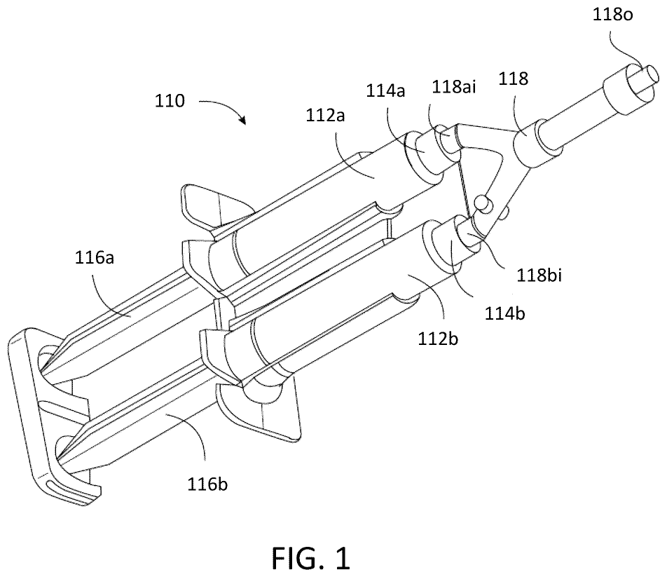

In particular embodiments, and with reference to FIG. 1, the system may include a delivery device 110 that comprises a double-barrel syringe, which includes a first barrel 112a having a first barrel outlet 114a, which first barrel contains the precursor fluid composition, a first plunger 116a that is movable in the first barrel 112a, a second barrel 112b having a second barrel outlet 114b, which second barrel 112b contains the fluid accelerant composition, and a second plunger 116b that is movable in the second barrel 112b. In some embodiments, the device 110 may further comprise a mixing section 118 having a first mixing section inlet 118ai in fluid communication with the first barrel outlet 114a, a second mixing section inlet 118bi in fluid communication with the second barrel outlet, and a mixing section outlet 118o.

In some embodiments, the delivery device may further comprise a cannula or catheter tube that is configured to receive precursor fluid and fluid accelerant compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the precursor fluid and fluid accelerant compositions are dispensed from the first and second barrels, whereupon the precursor fluid and fluid accelerant compositions mix and ultimately crosslink to form a crosslinked hydrogel, which is administered onto or into tissue of a subject. For example, the precursor fluid and fluid accelerant compositions may pass from the first and second barrels, into the mixing section via first and second mixing section inlets, whereupon the precursor fluid and fluid accelerant compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the precursor fluid composition may pass from the first barrel outlet into a first lumen of a multi-lumen catheter and the fluid accelerant composition may pass from the second barrel outlet into a second lumen of the multi-lumen catheter. In some embodiments the precursor fluid and fluid accelerant compositions may pass from the first and second lumen into a mixing section at a distal end of the multi-lumen catheter via first and second mixing section inlets, respectively, whereupon the precursor fluid and fluid accelerant compositions are mixed in the mixing section to form an admixture, which admixture exits the mixing section via the mixing section outlet.

In various embodiments, kits are provided that include a first reservoir containing a first composition as described herein, a second reservoir containing a second composition as described herein, and a third reservoir containing a third composition as described herein. The first, second and third reservoirs may independently be selected from, for example, vials, syringe barrels, autoinjectors and pumps. The kits may comprise one or more delivery devices for delivering the crosslinked hydrogel composition to a subject such as a double-barrel syringe like that described herein. The kits may comprise one or more of the following: a needle, a flexible tube (e.g., adapted to fluidly connect the needle to the syringe), a cannula, and an injectable liquid such as water for injection, normal saline or phosphate buffered saline. Whether supplied in a syringe, vial, or other reservoir, the crosslinked hydrogel composition may be provided in dry form (e.g., powder form) or in a form that is ready for injection, such as an injectable hydrogel form (e.g., a suspension of crosslinked hydrogel particles).

Regardless of the type of device that is used to mix the precursor fluid and fluid accelerant compositions or how the precursor fluid and fluid accelerant compositions are mixed, immediately after an admixture of the precursor fluid and fluid accelerant compositions is formed, the admixture is initially in a fluid state and can be administered to a subject (e.g., a mammal, particularly, a human) by a variety of techniques. Alternatively, the precursor fluid and fluid accelerant compositions may be administered to a subject independently and a fluid admixture of the precursor fluid and fluid accelerant compositions formed in or on the subject. In either approach, a fluid admixture of the precursor fluid and fluid accelerant compositions is formed and used for various medical procedures.

For example, the precursor fluid and fluid accelerant compositions or a fluid admixture thereof can be injected to provide spacing between tissues, the precursor fluid and fluid accelerant compositions or a fluid admixture thereof can be injected (e.g., in the form of blebs) to provide fiducial markers, the precursor fluid and fluid accelerant compositions or a fluid admixture thereof can be injected for tissue augmentation or regeneration, the precursor fluid and fluid accelerant compositions or a fluid admixture thereof can be injected as a filler or replacement for soft tissue, the precursor fluid and fluid accelerant compositions or a fluid admixture thereof can be injected to provide mechanical support for compromised tissue, the precursor fluid and fluid accelerant compositions or a fluid admixture thereof be injected as a scaffold, and/or the precursor fluid and fluid accelerant compositions or a fluid admixture thereof can be injected as a carrier of therapeutic agents in the treatment of diseases and cancers and the repair and regeneration of tissue, among other uses.

After administration of the compositions of the present disclosure (either separately as precursor fluid and fluid accelerant compositions that mix in vivo or as a fluid admixture of the precursor fluid and fluid accelerant compositions) a crosslinked hydrogel is ultimately formed at the administration location.

As seen from the above, the compositions of the present disclosure may be used in a variety of medical procedures, including the following, among others: a procedure to implant a fiducial marker comprising a crosslinked product of the precursor fluid and fluid accelerant compositions, a procedure to implant a tissue regeneration scaffold comprising a crosslinked product of the precursor fluid and fluid accelerant compositions, a procedure to implant a tissue support comprising a crosslinked product of the precursor fluid and fluid accelerant compositions, a procedure to implant a tissue bulking agent comprising a crosslinked product of the precursor fluid and fluid accelerant compositions, a procedure to implant a therapeutic-agent-releasing depot comprising a crosslinked product of the precursor fluid and fluid accelerant compositions, a tissue augmentation procedure comprising implanting a crosslinked product of the precursor fluid and fluid accelerant compositions, a procedure to introduce a crosslinked product of the precursor fluid and fluid accelerant compositions between a first tissue and a second tissue to space the first tissue from the second tissue.

The precursor fluid and fluid accelerant compositions or fluid admixtures of the precursor fluid and fluid accelerant compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the crosslinked hydrogel composition to a subject. Such systems may include one or more of the following: a syringe barrel, which may or may not contain a crosslinked hydrogel composition as described herein; a vial, which may or may not contain a crosslinked hydrogel composition as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel composition may be provided in dry form (e.g., powder containing crosslinked hydrogel particles) or in a form that is ready for injection, such as an injectable hydrogel form (e.g., a fluid suspension of crosslinked hydrogel particles).

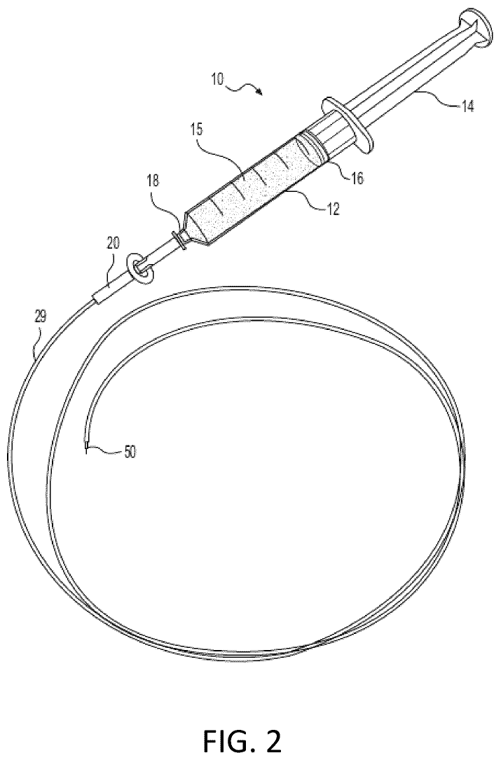

FIG. 2 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

As seen from the above, the crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

1. A system for forming a hydrogel composition that comprises (a) a first reservoir containing a reactive polymer that comprises a plurality of first reactive groups; (b) a second reservoir containing a multifunctional compound that comprises a plurality of second reactive groups, the second reactive groups reacting with the first reactive groups to form covalent bonds at a pH greater than 7.4; and (c) a third reservoir containing a biologically innocuous buffer having a pH in a range of 8 to 12.

2. The system of claim 1, wherein the first reactive groups are electrophilic groups and the second reactive groups are nucleophilic groups.

3. The system of claim 2, wherein the first reactive groups are cyclic imide ester groups and the second reactive groups are amine groups.

4. The system of claim 1, wherein the biologically innocuous buffer is selected from organic buffers, inorganic buffers, and non-nucleophilic polymeric buffers.

5. The system of claim 1, wherein the biologically innocuous buffer is an organic buffer that comprises a secondary or tertiary amine and does not comprise a primary amine.

6. The system of claim 1, wherein the biologically innocuous buffer is an inorganic buffer that selected from a bicarbonate buffer, a phosphate buffer, or a nanoclay-based buffer.

7. The system of claim 1, wherein the reactive polymer is a reactive multi-arm polymer that comprises a plurality of hydrophilic polymer arms comprising hydrophilic polymer segments and first reactive groups as end groups.

8. The system of claim 7, wherein the hydrophilic polymer segments are selected from polyalkylene oxide segments, polyester segments, polyoxazoline segments, polydioxanone segments, and polypeptide segments.

9. The system of claim 7, wherein the first reactive groups are linked to the hydrophilic polymer segments by a hydrolysable ester.

10. The system of claim 1, wherein the multifunctional compound comprises a plurality of —(CH₂)ₓ—NH₂ groups where x is 0, 1, 2 3, 4, 5 or 6.

11. The system of claim 1, wherein the multifunctional compound comprises a peptide oligomer that comprises from 2 to 10 lysine and/or ornithine amino acid residues.

12. The system of claim 1, further comprising a delivery device.

13. The system of claim 12, wherein the delivery device comprises a double barrel syringe.

14. The system of claim 1, further comprising one or more of a needle, catheter tube or cannula.

15. A system for forming a hydrogel composition that comprises (a) a first reservoir that contains a dry composition containing a reactive polymer that comprises a plurality of first reactive groups, (b) a second reservoir that contains a first fluid composition containing a multifunctional compound that comprises a plurality of second reactive groups, the second reactive groups reacting with the first reactive groups to form covalent bonds at a pH greater than 7.4, and (c) a third reservoir that contains a second fluid composition containing a biologically innocuous buffer having a pH in a range of 8 to 12.

16. The system of claim 15, wherein the first fluid composition containing the multifunctional compound is buffered to a pH ranging from 3 to 5.

17. A method of treating a subject using a system that comprises (i) a first reservoir that contains a dry composition containing a reactive polymer that comprises a plurality of first reactive groups, (ii) a second reservoir that contains a first fluid composition containing a multifunctional compound that comprises a plurality of second reactive groups, the second reactive groups reacting with the first reactive groups to form covalent bonds at a pH greater than 7.4, and (iii) a third reservoir that contains a second fluid composition containing a biologically innocuous buffer having a pH in a range of 8 to 12, the method comprising:
mixing the first fluid composition containing the multifunctional compound in the second reservoir with the dry composition containing the reactive polymer in the first reservoir to form a precursor fluid;
mixing the precursor fluid with the second fluid composition containing the biologically innocuous buffer in the third reservoir to form a fluid mixture; and
injecting the fluid mixture into or onto tissue of the subject whereupon a hydrogel forms in or on the tissue.

18. The method of claim 17, wherein the fluid mixture is injected from a device that simultaneously mixes the precursor fluid and the second fluid composition.

19. The method of claim 17, wherein the precursor fluid has a pH ranging from 3 to 5.

20. The method of claim 17, wherein the fluid mixture has a pH ranging from 7.4 to 12.
